(19) United States
(12) Patent Application Publication
(10) Pub. No.: US 2008/0319195 A1
(43) Pub. Date: Dec. 25, 2008

(54) Enantioselective Preparation of Benzimidazole Derivatives and Their Salts

(75) Inventors: Biao Jiang, Shanghai (CN); Xiao-Long Zhao, Shanghai (CN); Jia-Jia Dong, Shanghai (CN); Wan-Jun Wang, Shanghai (CN)

Correspondence Address: SMITH PATENT CONSULTING CONSULTING, LLC, 3309 DUKE STREET, ALEXANDRIA, VA, 22314, US

(73) Assignee: RATIOPHARM GMBH, Ulm (DE)

(21) Appl. No.: 12/158,450

(22) PCT Filed: Apr. 19, 2006
(86) PCT No.: PCT/EP06/03587

§ 371 Date: Jun. 20, 2008

(30) Foreign Application Priority Data
Dec. 22, 2005 (DE) 10 2005 061 720.4

(51) Int. Cl. C07D 401/12; C07D 235/28

(52) U.S. Cl. 546/183; 548/307.1; 546/273.7

(57) ABSTRACT

The invention relates to a new process for preparing benzimidazole derivatives having a chiral sulfoxide group in enantiomerically pure form or in a form in which one of the two enantiomers is present in an increased quantity over the other enantiomer. The invention likewise relates to a process for preparing the salts of the individual enantiomers of the benzimidazole derivatives with a chiral sulfoxide group. The invention relates in particular to a process for preparing the S-enantiomer of omeprazole (also known as esomeprazole) and the salts thereof, more particularly the zinc salt of the S-enantiomer of omeprazole. In the new process a prochiral sulfide is oxidized in an organic solvent with an oxidizing agent in the presence of a titanium(IV) complex.

The invention relates to a new process for preparing benzimidazole derivatives having a chiral sulfoxide group in enantiomerically pure form or in a form in which one of the two enantiomers is present in an increased quantity over the other enantiomer. The invention likewise relates to a process for preparing the salts of the individual enantiomers of the benzimidazole derivatives with a chiral sulfoxide group. The invention relates in particular to a process for preparing the S-enantiomer of omeprazole (also known as esomeprazole) and the salts thereof, more particularly the zinc salt of the S-enantiomer of omeprazole. The terms S-enantiomer of omeprazole and esomeprazole are used synonymously in this application.

Benzimidazole derivatives having a chiral sulfoxide group are known inhibitors of the gastric acid secretion, and many compounds of this group are used as pharmaceutical preparations for treating gastrointestinal diseases, in particular gastric ulcer. In this connection, the known active substances omeprazole, pantoprazole, lansoprazole and rabeprazole can be mentioned by way of example. On account of the sulfoxide group, the active substances are chiral so that the preparation of the compounds in an enantiomerically pure form is of interest. In particular, the S-enantiomer of omeprazole, i.e. the esomeprazole, is currently marketed on a large scale in the form of its magnesium salt.

The separation of substituted 2-(2-pyridinyl methyl sulfinyl)-1H-benzimidazoles into the individual enantiomers is described in DE 40 35 455, WO 94/27988, and WO 2004/002982, for example. These publications also relate in particular to the separation of omeprazole into its two enantiomers. The processes described in these publications use the racemate of the compounds and convert the racemate by means of an optically active compound into a diastereomer pair which can then be separated as usual. The isolation of an enantiomer from a mixture, enriched with this enantiomer, of two enantiomers of chiral benzimidazole compounds is also described in WO 97/02261. Such processes for separating a racemic mixture have a number of drawbacks since, on the one hand, the undesired enantiomer must usually be discarded and, on the other hand, the separation is connected with complex steps reducing the yield.

Correspondingly, there are a number of proposals in this field of how to produce the individual enantiomers of benzimidazole derivatives having a chiral sulfoxide group using a chiral synthesis.

In this connection, reference can be made to e.g. WO 96/17076, which discloses the microbial oxidation of the corresponding parochial sulfides into the individual enantiomers of the desired sulfoxide compounds, or WO 96/17077, which describes the microbial reduction of racemic sulfides into the desired sulfoxide stereoisomers. However, these processes are microbial processes, and it would be desirable to obtain a chemical process for the symmetric synthesis of the corresponding individual enantiomers of benzimidazole derivatives having a chiral sulfoxide group.

WO 96/002535 discloses a process in which a prochiral sulfide is reacted with an oxidant in the presence of a catalyst. The catalyst is a titanium complex having a diethyl tartrate as the bidentate chiral ligand. However, the disadvantage of the process is that it requires very specific reaction conditions. Thus, the reaction usually has to be carried out in the presence of a base and in a very specific order. For example, the titanium complex has to be reacted in the presence of the prochiral sulfide, and the reaction should be carried out at an elevated temperature and/or an increased reaction period. Furthermore, a very special oxidant, namely cumol hydroperoxide, is used in the processes of this publication.

A more recent process for the asymmetric synthesis of benzimidazole derivatives having a chiral sulfoxide group is described in WO 03/089408. This reaction is carried out in the presence of a base having a titanium or vanadium catalyst with a chiral monodentate ligand.

In general, the state of the art discloses a plurality of processes for the asymmetric oxidation of sulfides into optically active sulfoxides, and reference can be made to the publication ‘Journal of Organic Chemistry’ 1998, 63, 9392-9395, for example. However, it cannot be predicted whether one of the numerous, generally described processes for the asymmetric oxidation of sulfides into the optically active sulfoxides is also suited to produce the desired substituted benzimidazoles having a chiral sulfoxide group and is advantageous with respect to the known processes. This applies in particular to the asymmetric synthesis of the individual enantiomers of omeprazole, in particular esomeprazole.

Therefore, there is a need for further processes for the preparing individual enantiomers of benzimidazole derivatives having a chiral sulfoxide group, which do not show the disadvantages of the prior art.

The invention provides a process for the production of an optically active enantiomer or an enantiomer-enriched form of a compound of formula (I)

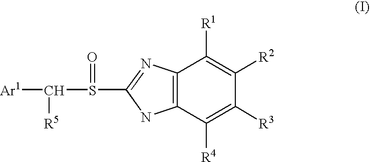

in which the residues R¹, R², R³, and R⁴ are independently hydrogen, alkyl, alkoxy, halogen, halogenalkoxy, alkylcarbonyl, alkoxycarbonyl, oxazolyl or halogenalkyl or adjacent residues R¹, R², R³, and R⁴ optionally form substituted ring structures, R⁵ represents a hydrogen atom or is joined with the residue Ar¹ to give a condensed ring system, and Ar¹ is a residue of formula

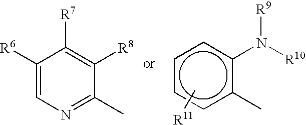

in which the residues R⁶, R⁷ and R⁸ are independently hydrogen, alkyl, alkylthio, alkoxy, halogen substituted alkoxy, alkoxyalkoxy, dialkylamino, piperidino, morpholino, halogen, phenylalkyl or phenylalkoxy or one of these residues is joined with the residue R⁵ to give a condensed ring system, the residues R⁹ and R¹⁰ are independently hydrogen, halogen or alkyl, and the residue R¹¹ is hydrogen, halogen, trifluoromethyl, alkyl, or alkoxy.

In the process according to the invention, a prochiral sulfide of formula (II)

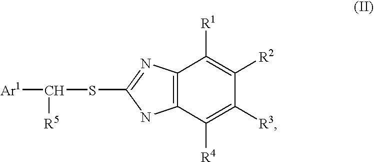

in which the residues R¹, R², R³, R⁴, R⁵, and Ar¹ are as defined above, is oxidized in an organic solvent with an oxidant in the presence of a catalyst. The process is characterized in that the catalyst is a titanium(IV) complex which can be obtained by reacting a titanium(IV) compound with a chiral bidentate (R,R)— or (S,S)-1,2-bis-arylethane-1,2-diol.

The chiral bidentate (R,R)— or (S,S)-1,2-bis-arylethane-1,2-diol is preferably a compound of general formula (III) or (III′)

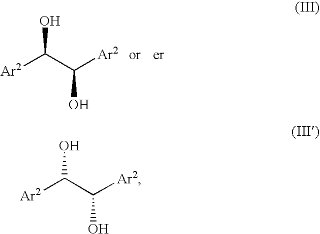

in which the residue Ar² is selected from

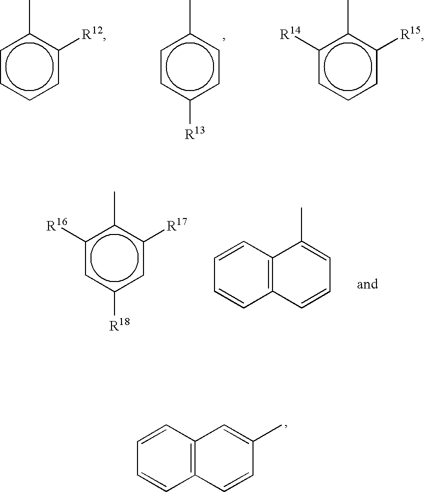

in which the residues R¹² to R¹⁸ are independently selected from hydrogen, alkyl, alkoxy, carboxylic acid ester residue, halogen, phenyl, trifluoromethyl and NO₂.

Based on the present application, alkyl is preferably a C₁-C₂₀, preferably a C₁-C₁₀, more preferably a C₁-C₆, alkyl residue, such as a methyl, ethyl, isopropyl, n-propyl, n-butyl, isobutyl, and tert.-butyl group.

Based on the present application, alkoxy is preferably an alkoxy residue having 1 to 20, more preferably 1 to 10, and in particular 1 to 6, carbon atoms, such as a methoxy, ethoxy, isopropoxy, n-propoxy, n-butoxy, isobutoxy, and tert.-butoxy group.

Based on the present application, halogen is a halogen atom, in particular a fluorine, chlorine, bromine, or iodine atom, fluorine atoms being particularly preferred.

Based on the present application, halogenalkoxy is preferably alkoxy as defined above which is substituted with one or several, in particular 1 to 5, more preferably 1 to 3, in particular 1, 2, or 3, halogen atoms, as defined above. The halogen atoms can be equal or differ and be located at one or more carbon atoms. It is preferred for the halogen atoms to be equal and (in so far as chemically possible) be bound to the same carbon atom, as in a CF₃ group, for example.

Based on the present application, an alkylcarbonyl residue is preferably alkyl as defined above which has a carbonyl functionality C═O.

Based on the present application, alkoxycarbonyl is preferably alkoxy as defined above which has a carbonyl group, C═O.

Based on the present application, aryl is preferably a phenyl or 1-naphthyl or 2-naphthyl group. Where appropriate, aryl can be substituted with one to three substituents, in particular with halogen atoms, nitro, alkyl and alkoxy, as defined above.

Based on the present application, alkylthio is preferably alkyl as defined above which has a thio group.

Based on the present application, alkoxyalkoxy is preferably alkoxy as defined above which is substituted with an alkoxy as defined above.

Based on the present application, dialkylamino is an amino group which is substituted with two alkyls as defined above.

Based on the present application, phenylalkyl and phenylalkoxy are alkyl and alkoxy, respectively, as defined above, which are substituted with a phenyl group.

Based on the present application, a carboxylic acid ester residue is preferably the residue of a carboxylic acid having 1 to 10 carbon atoms, preferably 1 to 6 carbon atoms, more preferably 1 to 4 carbon atoms.

Based on the present application arylalkyl is preferably alkyl as defined above which is substituted with an aryl as defined above.

If residues based on the present application may form ring structures or condensed ring systems, these residues preferably form a carbon ring having 5 to 10 carbon atoms, preferably 5, 6 or 7 carbon atoms, which may be substituted, where appropriate.

If based on the present application a unit may be substituted, it is preferably substituted with a halogen atom of a C₁-C₆ alkyl group or a C₁-C₆ alkoxy group as defined above, unless stated otherwise.

According to the invention it is particularly preferred for the compound of formula (I) to be a compound of formula

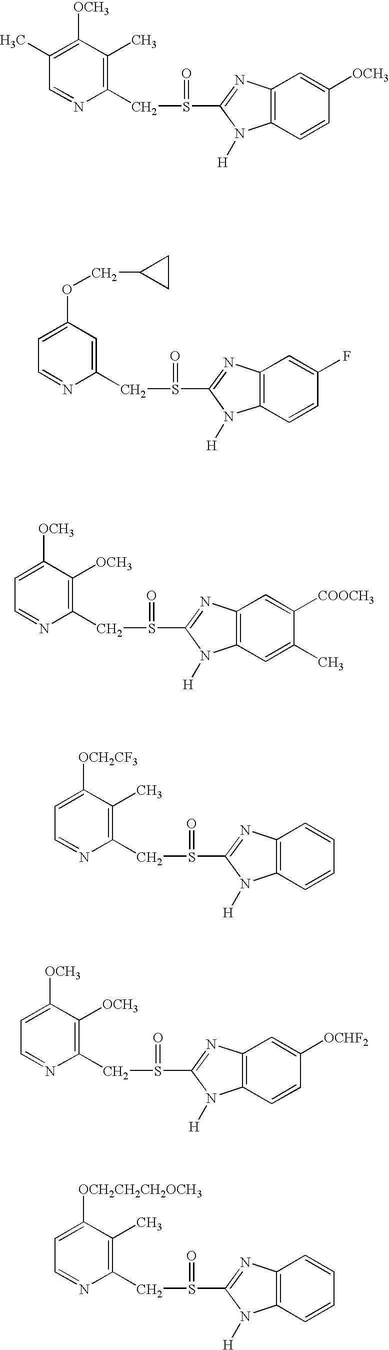

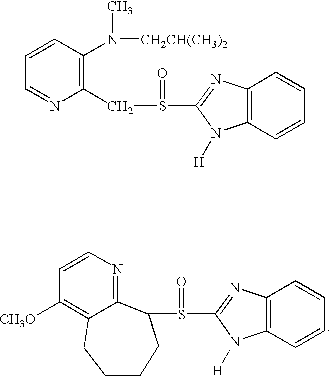

or

Most preferably, the compound of formula (I) is omeprazole, pantoprazole, lansoprazole, or rabeprazole according to the invention. Omeprazole is most preferred. In a particularly preferred embodiment, the invention therefore relates to a process for the production of an enantiomer of omeprazole and the salts thereof or a mixture of both enantiomers of omeprazole in which an enantiomer is present at a quantity increased with respect to the other enantiomer. In particular, the invention relates to a process for the production of esomeprazole and the salts thereof, in particular the zinc salt of esomeprazole.

The invention is further exemplified below, substantially by means of omeprazole. The below statements likewise apply to the other compounds of formula (I).

In the residues Ar², it is particularly preferred for the residue R¹² to be alkyl as defined above or halogen as defined above, in particular a bromine atom.

The residue R¹³ is particularly alkyl as defined above, halogen as defined above, in particular a bromine atom or alkoxy as defined above, more preferably an alkyl residue having 1 to 4 carbon atoms, or a bromine atom.

The residues R¹⁴ and R¹⁵ are preferably equal and selected from a hydrogen atom, halogen, alkyl, and alkoxy, as defined above.

Residues R¹⁶, R¹⁷, and R¹⁸ are also preferably equal and more preferably a hydrogen atom or alkyl as defined above.

Particularly preferred is the (R,R)— or (S,S)-1,2-bis-arylethyl-1,2-diol, the compound

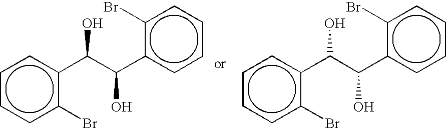

The (R,R)— or (S,S)-1,2-bis-arylethyl-1,2-diol, which is used as a chiral ligand of the titanium compound according to the invention, can be produced in manner known per se and is commercially available. In particular, these compounds can be obtained by asymmetric dihydroxylation of (E)-stilbene or the corresponding stilbene derivatives, as described in Chem. Rev. 1994, 94, 2483 or Chirality 13: 258-265 (2001), for example. Reference is made to the full contents of both publications as regards the production of the diols.

According to the invention the catalyst is formed in situ by reacting a suitable titanium compound, in particular a titanium(IV) alkoxide, preferably a titanium(IV) isopropoxide (Ti(1-PrO)₄), with the corresponding chiral diol of formula (III) or (III′). The titanium(IV) compound is reacted with the diol in an organic solvent, preferably in the presence of water, and a solvent the same as that subsequently used for the oxidation is preferably used for the catalyst production. In particular halogen substituted or unsubstituted alkyl or aryl hydrocarbons, such as methylene chloride, chloroform, carbon tetrachloride, hexane and toluene, are concerned in this connection. The most preferred organic solvent is toluene. The catalyst is preferably produced at a temperature ranging from 20° C. to 50° C., in particular ranging from 20° C. to 25° C. The catalyst is produced over a period of 1 to 60 minutes, preferably over a period of 10 to 20 minutes.

According to the invention it is preferred to initially produce the catalyst in situ and then the prochiral sulfide of formula (II), in particular the compound

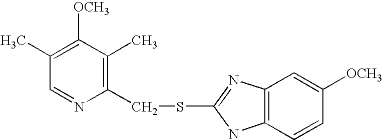

is added to the reaction mixture containing the catalyst, and the oxidant is subsequently added.

Alternatively, it is also preferred to produce the catalyst in the presence of the prochiral sulfide of formula (II), i.e. e.g. the titanium compound is initially added, then the prochiral sulfide of formula (II) is admixed and subsequently the chiral diol is inserted. Here, too, the oxidant is preferably added in a final step.

The subsequent oxidation is preferably carried out in the same solvent mixture in which the catalyst was also produced, i.e. also in the presence of water, in an organic solvent which is preferably selected from halogen substituted or unsubstituted alkyl and aryl hydrocarbons, such as methylene chloride, chloroform, carbon tetrachloride, hexane and toluene, preferably toluene.

The enantioselective catalytic reaction is preferably carried out at a temperature ranging from −78° C. to 25° C., more preferably at about −20° C. to 0° C., in particular at about −20° C. The enantioselective catalytic oxidation usually lasts 2 to 24 hours, preferably 12 to 18 hours.

Every common oxidant can be used according to the invention; however, the oxidant is preferably hydrogen peroxide, an alkyl hydroperoxide or an arylalkyl hydroperoxide, with tert-butylhydroperoxide being particularly preferred. The oxidant is preferably not cumol hydroperoxide.

The enantioselective catalytic oxidation according to the invention is preferably carried out without the addition of a base.

In the method according to the invention, the quantities of catalyst and prochiral sulfide of formula (II) are preferably chosen such that the molar ratio of chiral, bidentate ligand to prochiral sulfide of formula (II) ranges from 0.02:1 to 0.4:1, and is more preferably about 0.1:1. According to the invention the molar ratio of titanium compound to sulfide of formula (II) preferably ranges from 0.01:1 to 0.2:1, and is more preferably about 0.05:1.

According to the invention the production of the catalyst and also the subsequent enantioselective catalytic oxidation are preferably carried out in the presence of water, the molar ratio of water to prochiral sulfide of formula (II) preferably ranging from 0.01:1 to 2:1 and being more preferably about 1:1.

The amount of oxidant used is not critical; the ratio of oxidant to prochiral sulfide of formula (II) preferably ranges from 0.5:1 to 3:1 and is preferably about 2:1.

Having carried out the enantioselective oxidation of the prochiral sulfide of formula (II) into the chiral sulfoxide of formula (I), the processing of the reaction mixture is not particularly critical; yet is was found that after a special processing method the sulfoxide of formula (I), in particular the esomeprazole, accumulates in the basic form which can subsequently be converted into its salts in a particularly easy way.

Having carried out the enantioselective catalytic oxidation of the compound of formula (II) into the compound of formula (I), the reaction mixture is preferably treated with an aqueous, basic solution according to the invention. The aqueous, basic solution is preferably an aqueous ammonia solution. Having added the ammonia solution, an acid is introduced which may be the aqueous solution of an inorganic acid or an organic acid, an organic acid being preferred and acetic acid being particular preferred. The pH adjusted is preferably within the range of 5 to 8, more preferably within the range of 6 to 7.5. The resulting, aqueous solution is extracted with an organic solvent, with halogen substituted or unsubstituted alkyl or aryl hydrocarbons and ketones, such as methylene chloride, chloroform, carbon tetrachloride, hexane, toluene, acetone, butanone and methyl isobutyl ketone being particularly preferred. Other conventional organic extracting agents can also be used. The preferred solvent for the extraction is methyl isobutyl ketone.

It has surprisingly been found that the compound of formula (I), in particular (S)-omeprazole (esomeprazole), precipitates in pure form when the organic extracting agent is cooled. The extracting solution is preferably cooled to a temperature ranging from −78° C. to 25° C., more preferably from −20° C. to 0° C., e.g. to about −10° C., and the desired enantiomer of the compound of formula (I) precipitates as a solid in the form of the free base.

In this way, in particular the S-enantiomer of omeprazole can readily be obtained in very good yield and optical purity. If the resulting product still contains residues of the undesired enantiomer of the compound of formula (I), these can be separated as usual to raise the optical purity.

The desired enantiomer usually accumulates as a solid as a mixture of amorphous and crystalline product, in particular when esomeprazole is produced.

Since as a result of the preferred processing the desired isomer of the compound of formula (I) accumulates in the form of the free base as a solid in the process according to the invention, it can be converted into a salt in a particularly favorable way. This is an advantage with respect to the prior art process in which a certain salt of esomeprazole can only be produced in a complicated way, as described in WO 98/28294, for example, namely by dissolving an alkaline salt of esomeprazole in water, extracting the neutral esomeprazole with an organic solvent by lowering the pH using a water-soluble acid, evaporating the product to give a strongly concentrated solution and adding a non-solvent so as to precipitate the esomeprazole in a basic form as a solid. According to the process of the invention, the free base is readily obtained directly when the reaction mixture is processed in a suitable way after the enantioselective, catalytic oxidation of the corresponding sulfide into the sulfoxide. The free base can then be converted into a desired salt as usual, the salts being not particularly limited. In this connection, in particular the sodium, magnesium, lithium, potassium, calcium, and quaternary ammonium salt can be mentioned, but also the piperidine salt and in particular the zinc salt. It is particularly preferred to produce the zinc salt of esomeprazole according to the invention by treating the esomeprazole with a suitable zinc source. Preferred zinc sources are zinc acetate, zinc bromide, zinc carbonate hydroxide, zinc chloride, zinc trifluoromethane sulfonate, zinc nitrate, diethyl zinc and zinc sulfate, with diethyl zinc and zinc chloride—in particular diethyl zinc—being particularly preferred.

According to the invention, the most preferred embodiment therefore provides a process for the production of esomeprazole or a salt of esomeprazole, in particular the zinc salt of esomeprazole, which comprises the following steps:
a) (R,R)-1,2-bis-arylethyl-1,2-diol, in particular (R,R)-1,2-bis-(2-bromophenyl)ethane-1,2-diol, is added with a titanium(IV) alkoxide, in particular with titanium tetraisopropoxide, to an organic solvent;
b) water is admixed to this reaction mixture;
c) the corresponding sulfide of general formula (II), in particular compound

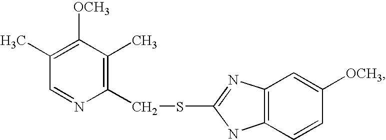

is admixed to the reaction mixture of step b);
d) the oxidant, in particular an alkyl or arylalkyl hydroperoxide, most preferably dibutyl hydroperoxide, is added to this mixture;
e) an aqueous, basic solution, in particular an aqueous ammonia solution, is admixed;
f) an acid, in particular an organic acid, such as acetic acid, is added to the mixture, preferably up to a pH ranging from 5 to 8, more preferably from 6 to 7.5;
g) the aqueous mixture is extracted with a suitable organic solvent;
h) the organic solvent is cooled, and the precipitated enantiomer of the compound of formula (I), in particular esomeprazole, is filtered off, and
i) where appropriate, converted into a salt, in particular a zinc salt.

The following examples explain the invention.

### EXAMPLE 1

### Preparation of S-Omeprazole

Titanium tetraisopropoxide (4.5 mg, 0.016 mmol) was added to a solution of (R,R)-1,2-bis-(2-bromophenyl)ethane-1,2-diol (12 mg, 0.032 mmol) in toluene (2 ml) at 25° C. The solution was stirred for 10 minutes, water (5.7 mg, 0.32 mmol) was added, and the solution was then stirred for another 10 minutes. 5-Methoxy-2-[[(4-methoxy-3,5-dimethyl-2-pyridinyl)-methyl]thio]-1H-benzimidazole (105 mg, 0.32 mol) was subsequently added to the solution, and the temperature was adjusted to −20° C. Thereafter, t-butyl hydroperoxide (70%, 96 μl, 0.064 mmol) was slowly added. After 12 hours at −20° C., the solution was extracted three times with aqueous ammonium hydroxide (12.5% NH₃, 3×5 ml). Thereafter, methyl isobutyl ketone (5 ml) was added to the combined aqueous extracts. Then, the pH of the aqueous phase was adjusted using acetic acid, the aqueous phase was separated and extracted with an additional amount of methyl isobutyl ketone (5 ml). The organic solution was cooled to −10° C. over night, and the neutral form of S-omeprazole was precipitated as a solid to obtain the title compound (99 mg, 90% yield). The enantiomeric excess of S-omeprazole was 94%. Purification using methyl isobutyl ketone yielded S-omeprazole, and the enantiomeric excess was >99%.

### EXAMPLE 2

### Production of R-omeprazole

Titanium tetraisopropoxide (4.5 mg, 0.016 mmol) was added to a solution of (S,S)-1,2-bis-(2-bromophenyl)ethane-1,2-diol (12 mg, 0.032 mmol) in toluene (2 ml) at 25° C. The solution was stirred for 10 minutes, water (5.7 mg, 0.32 mmol) was added, and the solution was stirred for another 10 minutes. 5-methoxy-2-[[(4-methoxy-3,5-dimethyl-2-pyridinyl)-methyl]thio]-1H-benzimidazole (105 mg, 0.32 mol) was then added to the solution, and the temperature was adjusted to −20° C. Thereafter, t-butyl hydroperoxide (70%, 96 μl, 0.064 mmol) was slowly added. After 12 hours at −20° C., the solution was extracted three times with aqueous ammonium hydroxide (12.5% NH₃, 3×5 ml). Thereafter, the methyl isobutyl ketone (5 ml) was added to the combined aqueous extracts. After this, the pH of the aqueous phase was adjusted using acetic acid, the aqueous phase was separated and extracted with an additional amount of methyl isobutyl ketone (5 ml). The organic solution was cooled to −10° C. over night, and the neutral form of R-omeprazole was precipitated as a solid to obtain the title compound. The enantiomeric excess of R-omeprazole was 93%.

### EXAMPLE 3

### Production of Esomeprazole Zinc

Esomeprazole (1 g, 2.9 mmol) was dissolved in 10 ml tetrahydrofuran while stirring for 5 hours, and 2.9 ml diethyl zinc (1 M solution in hexane) were slowly added. The resulting mixture was stirred at ambient temperature overnight. 10 ml distilled water were added, and the precipitate formed was filtered off and washed with distilled water. 1 g (91%) of the title compound was obtained.

### EXAMPLE 4

### Production of the Catalyst Ligand

(E)-2,2-dibromostilbene

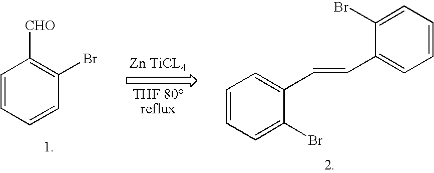

4.4 ml (7.4 g, 40 mmol) of a yellow slurry of titanium(IV) chloride in 150 ml tetrahydrofuran were stirred in an ice bath under nitrogen by means of a magnetic stirrer. 5 g (77 mmol) zinc dust were carefully added. Then, 7 g (38 mmol) aldehyde 1 in 50 ml tetrahydrofuran were admixed, and the mixture was refluxed for 8 hours. The cooled reaction mixture was poured into 1 M dilute, aqueous hydrochloric acid, and the product was extracted using hexane. The combined extracts were washed with water and (common) salt solution, dried with sodium sulfate, filtrated, and the filtrate was rotary evaporated, 6.2 g (97%) of 2 forming. 2 was obtained as white needles by recrystallization from a mixture of 5% toluene and 95% ethanol.

### (1R,2R)-1,2-bis-(2-bromophenyl)ethane-1,2-diol

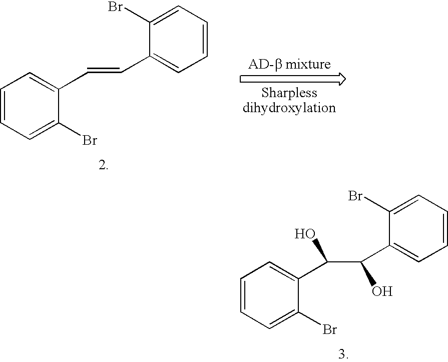

Methane sulfonamide (3.39 g, 0.0419 mol) and AD-mix-β (50.2 g) were added to a 1-liter three-neck flask containing water (180 ml) and 2-methylpropane-2-ol (180 ml). The mixture was stirred using a mechanical stirrer until all solids had been dissolved. The flask was then cooled to 0° C., and dibromostilbene 2 (12.0 g, 32.3 mmol) was added. The reaction mixture was vigorously stirred for 72 hours and kept between 0 and 3° C. Then, anhydrous sodium sulfide (54 g, 0.439 mol) was added, and the mixture was stirred overnight. Dichloromethane (350 ml) was added, and the phases were separated. The aqueous layer was extracted using dichloromethane (2×175 ml), and the combined organic layers were washed with 2 M KOH (30 ml), dried (MgSO₄), and volatile substances were evaporated at a reduced pressure. The residue was purified using flash chromatography by elution with ether-hexane and then recrystallized (hexane-dichloromethane, 1.1:1, 92 ml), the diol 3 (12.5 g, 94%) being obtained as needles.

1. A process for the production of optically active enantiomers or an enantiomer-enriched form of a compound of formula (I) 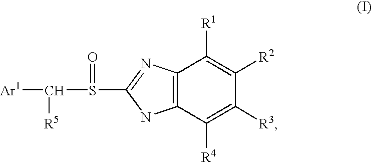
wherein:
the residues R¹, R², R³ and R⁴ are independently selected from the group consisting of hydrogen, alkyl, alkoxy, halogen, halogenalkoxy, alkylcarbonyl, alkoxycarbonyl, oxazolyl and trifluoroalkyl or adjacent residues R¹, R², R³ and R⁴ form substituted ring structures,
R⁵ represents a hydrogen atom or is connected with the residue Ar¹ to give a condensed ring system, and Ar¹ is a residue of formula 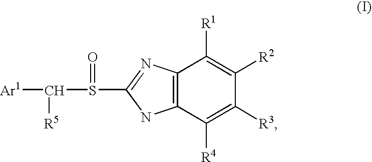
in which the residues R⁶, R⁷ and R⁸ are independently hydrogen, alkyl, alkylthio, alkoxy, halogen substituted alkoxy, alkoxyalkoxy, dialkylamino, piperidino, morpholino, halogen, phenylalkyl or phenylalkoxy, or one of said residues is joined with the residue R⁵ to give a condensed ring system, residues R⁹ and R¹⁰ are independently hydrogen, halogen or alkyl, and residue R¹¹ is hydrogen, halogen, trifluoromethyl, alkyl or alkoxy,
said process comprising the step of oxidizing a prochiral sulfide of formula (II) 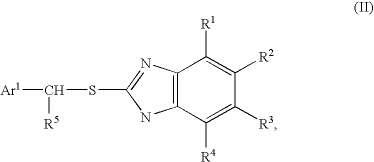
in which residues R¹, R², R³, R⁴, R⁵ and Ar¹ are as defined above, in an organic solvent with an oxidant in the presence of a catalyst, wherein the catalyst is a titanium(IV) complex which can be obtained by reacting a titanium(IV) compound with a chiral, bidentate (R,R)— or (S,S)-1,2-bis-arylethane-1,2-diol.

2. The process according to claim 1, wherein the compound of formula (I) has a formula selected from the group consisting of: 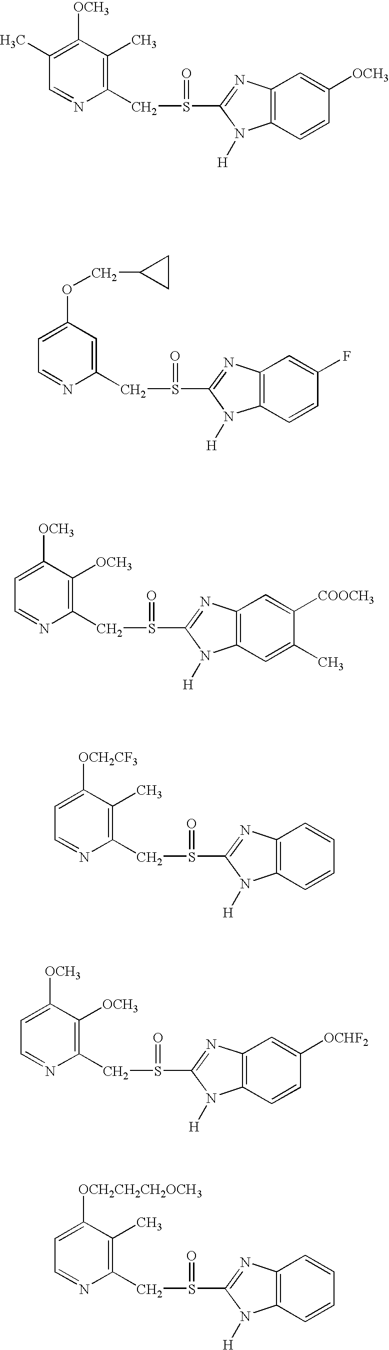 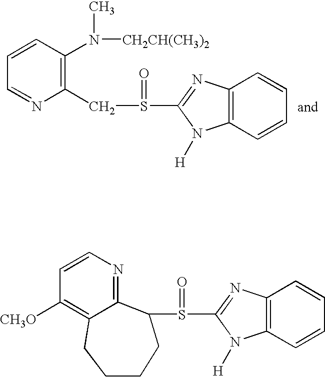
and the compound of formula (II) represents the corresponding prochiral sulfide.

3. The process according to claim 2, wherein the optically active enantiomers of the enantiomer-enriched form of a compound of formula (I) produced comprises the S-enantiomer of omeprazole or a mixture of the S- and R-enantiomers of omeprazole in which the omeprazole S-enantiomer is enriched.

4. The process according to claim 3, comprising the further step of reacting the S-enantiomer of omeprazole with a zinc source to give the zinc salt of the S-enantiomer of omeprazole.

5. The process according to claim 1, wherein the chiral, bidentate (R,R)— or (S,S)-1,2-bis-arylethane-1,2-diol is a compound of general formula (III) or (III′) 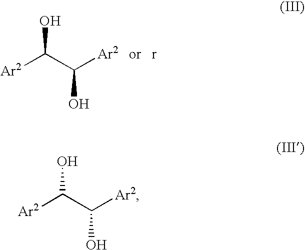
in which the residue A² is selected from 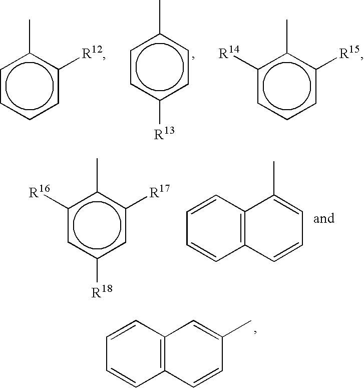
in which the residues R¹² to R¹⁸ are independently selected from the group consisting of hydrogen, alkyl, alkoxy, carboxylic acid ester residue, halogen, phenyl, trifluoromethyl and NO₂.

6. The process according to claim 5, wherein R¹⁴ and R¹⁵ are independently selected from the group consisting of hydrogen, alkyl, alkoxy and halogen, and the residues R¹⁶, R¹⁷ and R¹⁸ are independently selected from the group consisting of hydrogen and alkyl.

7. The process according to claim 6, wherein the residues R¹⁴ and R¹⁵ are equal and the residues R¹⁶, R¹⁷ and R¹⁸ are equal.

8. The process according to claim 7, wherein the residue R¹² is a bromine atom.

9. The process according to claim 8, wherein the (R,R)— or (S,S)-1,2-bis-aryl-1,2-diol is a compound of formula

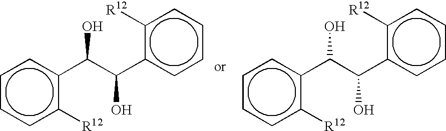

10. The process according to claim 1, wherein the titanium(IV) compound is an alkoxide of titanium(IV).

11. The process according to claim 10, wherein the titanium compound is the isopropoxide of titanium(IV).

12. The process according to claim 1, wherein the ratio of chiral, bidentate ligand to prochiral sulfide of formula (II) is within the range of 0.1:1.

13. The process according to claim 1, wherein the molar ratio of titanium(IV) alkoxide to prochiral sulfide of formula (II) is within the range of 0.05:1.

14. The process according to claim 1, wherein the reaction is carried out in the presence of water.

15. The process according to claim 1, wherein the oxidant is hydrogen peroxide, an alkyl hydroperoxide or an arylalkyl hydroperoxide.

16. The process according to claim 1, wherein the catalyst is produced by reacting the chiral ligand with the titanium(IV) alkoxide in an organic solvent before the prochiral sulfide of formula (II) is added to the reaction mixture.

17. The process according to claim 1, wherein the oxidation is carried out at about −20° C. over a period of 12 to 18 hours.

18. The process according to claim 1, comprising the following steps:
a) adding a mixture of the chiral, bidentate (R,R)— or (S,S)-1,2-bis-arylethane-1,2-diol with the titanium(IV) alkoxide in the presence of an organic solvent,
b) adding water to the mixture of step a),
c) adding the prochiral sulfide of formula (II) to the reaction mixture of step b),
d) adding the oxidant to the reaction mixture of step c),
e) adding aqueous ammonia to the reaction mixture of step d),
f) adding an acid to the aqueous mixture of step e),
g) extracting the aqueous mixture using an organic solvent,
h) cooling the organic solvent and filtrating the precipitated enantiomer of the compound of formula (I), and
i) where appropriate, converting the desired isomer of the compound of formula (I) into the zinc salt.
